(19) United States
(12) Patent Application Publication
(10) Pub. No.: US 2023/0056896 A1
(43) Pub. Date: Feb. 23, 2023

(54) COVER FOR A PROBE OF A CONTACT OPHTHALMOLOGICAL APPARATUS

(71) Applicant: CATS TONOMETER, LLC, Tucson, AZ (US)

(72) Inventor: Sean J. McCafferty, Tucson, AZ (US)

(73) Assignee: CATS TONOMETER, LLC, Tucson, AZ (US)

(21) Appl. No.: 17/889,713

(22) Filed: Aug. 17, 2022

Related U.S. Application Data
(60) Provisional application No. 63/252,472, filed on Oct. 5, 2021.
(60) Provisional application No. 63/242,752, filed on Sep. 10, 2021.
(60) Provisional application No. 63/235,058, filed on Aug. 19, 2021.
(60) Provisional application No. 63/235,026, filed on Aug. 19, 2021.
(60) Provisional application No. 63/235,024, filed on Aug. 19, 2021.

(51) Int. Cl. A61B 8/00; A61B 3/16; A61B 8/10

(52) U.S. Cl. CPC A61B 8/4422; A61B 3/16; A61B 8/10; A61B 2562/247

(57) ABSTRACT

A disposable biocompatible (and preferably hypoallergenic) flexible cover for use with a tip of a probe of a contact ophthalmological instrument. The outer surface of the tip (at the closed end) of the cover is configured to match the surface of the cornea, while its inner surface is preferably dimensioned to be substantially congruent with the surface of the tip of the probe with which the cover is used. A method for use of the same.

### CROSS-REFERENCE TO RELATED APPLICATIONS

This patent application claims priority from and benefit of the U.S. Provisional Pat. Applications Nos. 63/235,058 filed on Aug. 19, 2021; 63/235,024 filed on Aug. 19, 2021; 63/235,026 filed on Aug. 19, 2021; 63/242,752 filed on Sept. 10, 2021; and 63/252,472 filed on Oct. 05, 2021. The disclosure of each of the above-identified patent documents is incorporated by reference herein.

### TECHNICAL FIELD

The present invention relates generally to the field of ophthalmological instruments such as contact tonometers or pachymeters, for example, and more specifically to a hypoallergenic probe tip cover for such a contact ophthalmological device that employs a probe tip with a curved eye-contacting surface.

### RELATED ART

A multitude of reliable instruments are being currently used in ophthalmology including, to name just a few, a tonometer (used for testing intraocular pressure) and a pachymeter (used for measurement of thickness of the cornea prior to refractive surgery, for example), both of which instruments are advantageous in screening for patients suspected of developing glaucoma, for example. Glaucoma is characterized by \increase in pressure within the eye, but because the patient seldom experiences any symptoms until major damage occurs, regular testing is essential to detect glaucoma in the early state before the retinal field is seriously diminished, and ocular nerve damage has occurred.

Utilizing “contact” types of such devices - especially those that are hand-held -requires at least touching the eye surface directly with the probe of a device and, in some cases, pressing the tip against the cornea into the eye thereby indenting a portion of the cornea. (In this context, the probe of the contact instrument is understood to be that protruding part of it which is juxtaposed and/or cooperated with an eye under test. In some of related art documents, such probe portion is also and/or interchangeably referred to as a “tip” - for example, in the case of using the Goldmann type tonometer.)

A disadvantage of the use of an eye-contacting ophthalmological device is apparent from the very nature of its operation: such devices must touch the eye, and therefore, poses a risk of transferring various pathogens from one patient to another, or from patient to health care provider. While various methods have been utilized to reduce the transmission risk - including sterilization of the probe tip between examinations, the use of sterile gloves by the tester (to reduce a chance of recontamination of a tip/probe portion of the device that may occur when reinserting the probe into the probe holder of the device), and wiping of the probe tip surface, to name just a few - these precautions were proven to be insufficient to reliably remove pathogens and contaminations. For these reasons, a more preferred method has been to fit the probe, or contact tip, of a contact ophthalmological device with a protecting cover during the measurement process, at least in order to prevent the detrimental transmission of pathogens. Such cover can be made disposable and is often formed from materials such as natural latex rubber or various hypoallergenic materials, as discussed, for example, in US 7,287,856 (which considers a typical cover that is dimensioned for use with a probe of a contact tonometer and structured to have a substantially and overall convex -as viewed from outside of the unfolded cover- area of the tip or closed end of the cover).

Meanwhile, recently it has been discovered and experimentally proven that, to provide more meaningful and less erroneous measurements performed with the use of a contact ophthalmological instrument, the probe (tip) of such a device has to have a curved front surface. (See, for example, US D775,736; US 10,463,251; US 11,026,576; WO 2016/167827; serial numbers 63/235,024, 63/235,026, 63/242,752, and 63/252,472. The disclosure of each of these patent documents is incorporated herein by reference). A person of skill will readily appreciate that in such a case the use of a conventionally-structured cover - for example, a cover of the type discussed in US 7,287,856 (the disclosure of which is incorporated herein by reference) - during the measurement of, for example, intraocular pressure will substantially negate at least some if not all advantages that would otherwise be provided by the curved surface of the tip/probe of the ophthalmological device at least because a space, gap, and/or air bubble would remain present between the inner surface of the conventionally dimensioned cover and the cornea-contacting curved surface of the probe/tip, the presence of which quite possibly introduces additional unknown and/or non-correctable errors.

### SUMMARY OF THE INVENTION

Embodiments of the invention provide an article of manufacture that includes a hypoallergenic disposable probe tip cover for a contact ophthalmological instrument, which cover contains a thin film of material having an inwardly-curved tip or end (as observed from looking at the closed end of the cover from the outside) and which in at least one case is characterized by ultimate elongation parameter of from about 500% to about 1000%, a tensile strength of from about 1000 to about 5500 psi and a modulus of elasticity at 100% strain of from about 50 to about 2000 psi.

In at least one implementation, the invention provides a hypoallergenic disposable cover is judiciously structured to be used with the probe (of at least a contact tonometer or a contact pachymeter) that has a bi-curved front surface (which surface may be configured as a surface of a single material component or as a combination of surfaces of multiple material components forming the probe). Such cover includes the above-identified thin film, which - when installed onto the probe tip - creates a barrier against microorganisms from the eye of a patient to the probe tip during a tonometric examination while allowing and not impeding measurement of intraocular pressure through the cover. The effect of not creating measurement errors during the measurement with the use of the cover on top of the probe is achieved as a result of the surface of the closed end of the cover being substantially congruous with the surface of the tip of the probe of the contact instrument. Alternatively or in addition, the subject hypoallergenic disposable cover formed from a film selected from the group of materials consisting of polyurethane, polyethylene, polypropylene, polyisoprene, polychloroprene, nitrile, and silicone. In at least one related case, the embodiment may include a hypoallergenic disposable cover formed from cis-1,4-polyisoprene. Alternatively or in addition, the hypoallergenic disposable tip cover comprises a ring bead that is dimensioned to fit into a groove on the probe of the contact tonometer to hold the tip cover in place.

In at least one embodiment, the article of manufacture includes a flexible thin film tubular body having an open and a closed end, a tip portion defining the closed end of the body, and a wall portion connecting the closed end with the open end (here, the tip portion includes a first central area that has an inner surface and an outer surface, the outer surface being concave). The article is configured to create a barrier, when installed onto the probe tip, to transfer of microorganism between an eye of a patient and the tip of a probe of the contact instrument during a contact ophthalmological examination while, at the same time, not impeding a measurement of a target parameter of the eye through such cover. The configuration of the article is such that (i) the inner surface is either convex or substantially planar as viewed internally to tubular body from the open end; or the inner surface is convex; and that (ii) the outer surface is dimensioned to substantially conform to a corneal surface of an eye. In substantially any embodiment, the open end may be complemented with a circumscribing retention ring portion. In at least one - and, preferably, in every embodiment - the wall portion may be dimensioned to secure the cover to the contact ophthalmological instrument. When the retention ring portion is present, such retention ring portion may be configured to fit into a corresponding groove of said contact instrument. Alternatively or in addition, and substantially in every embodiment, the tip portion of the article may include a second peripheral area having an inner surface that is concave as viewed from the open end, such second peripheral area circumscribing the first central area.

Embodiments additionally provide a method that includes - while using an embodiment of the article of manufacture identified above - a step of removably covering a tip of a probe of a contact ophthalmological instrument with such article to spatially-coordinate the first central area with a front surface of the tip of a probe, and a step of bringing the tip of the probe in indirect contact with the cornea of an eye through a layer of the thin film body of the article at the first central area such that an outer surface of the first central area is in physical contact with the cornea while the tip of the probe is in physical contact with an inner surface of the first central area.

In at least one case, the step of bringing is that of bringing substantially congruent with one another (that is, substantially identical in form; substantially coinciding when superimposed) inner surface of the first central area and the front surface of the tip of the probe in contact with one another. Alternatively or in addition, the step of removably covering includes creating a barrier against microorganisms from the eye to the tip of a probe during the indirect contact while allowing a measurement of a target parameter of the eye through the cover substantially without impeding such measurement. At least one embodiment of the method additionally includes a step of performing a measurement of an intraocular pressure of an eye while pressing the tip of the probe and the cover into the cornea; or a step of performing a measurement of a thickness of the cornea by transmitting ultrasound waves through the cover while the covering remains in contact with the cornea.

### BRIEF DESCRIPTION OF THE DRAWINGS

The invention will be more fully understood by referring to the following Detailed Description of Specific Embodiments in conjunction with the Drawings, of which:

FIGS. 1A and 1B provide schematics of ophthalmological probes having a curved eye-contacting surface. FIG. 1A: a perspective view of a replaceable probe of Goldmann-type tonometer. FIG. 1B: schematic cross-sectional view of a curved-surface tip configured for use with a portable tonometer (such as a Tono-Pen®, for example). FIG. 1C presents schematically a related embodiment.

FIG. 2 illustrates schematically, in a cross-sectional view, a curved front surface of a tip of an ophthalmological instrument.

FIG. 3 provides, in a cross-sectional view, additional structural details of a specific embodiment of the ophthalmological probe for use with which an embodiment of the invention is configured.

FIG. 4 is a cross-sectional view of an embodiment probe tip cover of the invention.

FIG. 5 is a top view of the embodiment of the probe tip cover of the invention.

FIG. 6 is a perspective view of a portable contact tonometer apparatus with the probe tip covered with an embodiment of a cover.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another.

### DETAILED DESCRIPTION

In accordance with preferred embodiments of the present invention, methods and apparatus are disclosed for protecting a curved-front-surface tip or probe of an ophthalmological instrument such as contact tonometer or a contact pachymeter during measurements from biological contaminations while, at the same time, not impeding the measurements and/or not introducing measurement errors by so protecting.

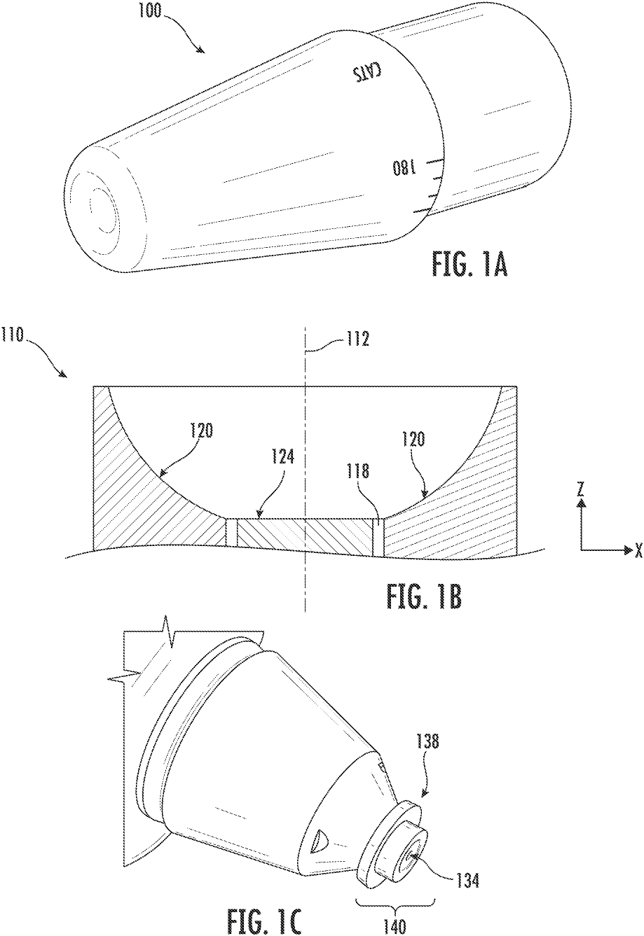

FIGS. 1A, 1B and 1C illustrate curved front surfaces of several related implementations of a probe of interest, each configured for use with an ophthalmological instrument. FIG. 1A depicts an embodiment of a probe that can be utilized in a contact tonometer such as Goldmann applanation tonometer (see, for example, US 10,463,251 and/or US 11,026,576 describing io detail the principle of operation of the embodiment 100 and its operational advantages), while FIG. 1B illustrates schematically the structure (as shown - rotationally symmetric about the axis 112) of the very tip of a probe configured for use with a portable tonometer of a Tono-Pen® variety (see, for example, U.S. Provisional Pat. Application 63/242,752). FIG. 1C presents schematically an embodiment 130 of the probe of a portable tonometer, the tip 134 of which has been modified by having it complemented with a “cap” 138.

In reference to the sketch of FIG. 1B, the embodiment 110 is shown to include a front surface 124 of the transducer of the embodiment and remains substantially planar; the front surface 120 of the outer portion of the tip 110 is inwardly curved such as to be merging with the surface 124 in a substantially tangentially-parallel fashion when the device is “off”. It is understood, therefore, that the front surface of the tip 110 contains now two surface portions, 120 and 124, one of which is curved and the other is planar, and which are tangentially-parallel with one another along the perimeters of the surface portions 120, 124 and/or corresponding edges of these surfaces. (And, when the annularly-shaped gap or opening 118 is identified between the surface 120, 124, these surfaces 120, 124 are made tangentially-parallel to one another substantially at every point across such opening.) In conventionally-attended measurement procedure, at least the surface 120 - and preferably, both the surface 120 and the surface 124 - are brought in contact with the cornea of an eye subject to a tonometric measurement (whether directly or indirectly, while separated from the cornea by an intermediate material). After, the portable tonometer of which the tip 110 is a part, is operated in a conventional fashion to measure the intraocular pressure (IOP) with the use of the transducer having the front surface 124.

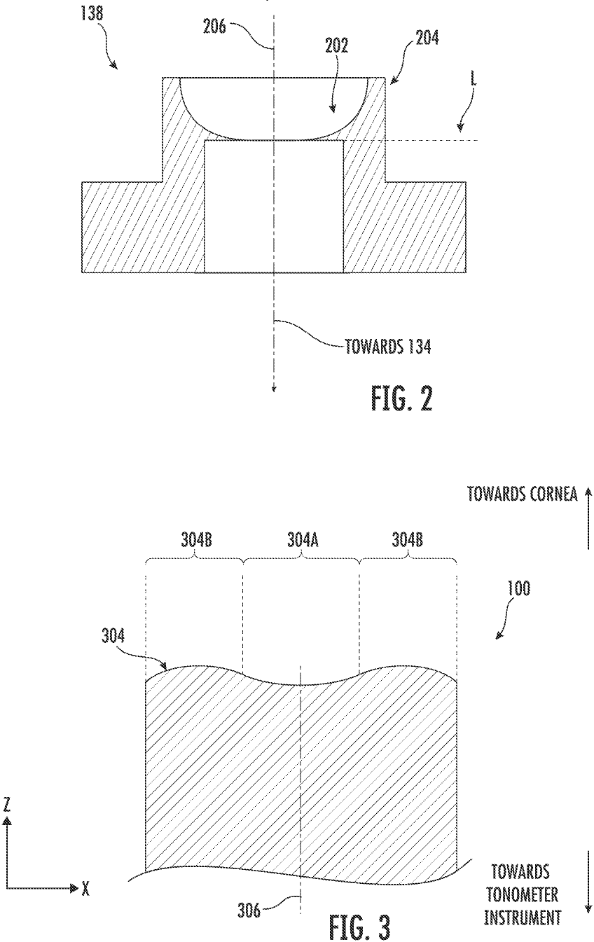

In reference to FIG. 1C, a rotationally-symmetric crown or cap 138 (separately illustrated schematically in FIG. 2 in a cross-section) has an inwardly caved from surface 202 and can be configured to fittingly and removably slid over the existing tip 134 of the probe 130 (for example, of the Avia Tonopen, as shown) such as to have the rim or ridge 204 of the crown be appropriately raised above the level L defined by the substantially planar cornea-contacting surface of the existing tip 134 (and, therefore, over the flat surface of the original tip 134 when mated with it.) The curved front surface of the cap / crown 138 is dimensioned to be tangentially parallel to the plane that is drawn perpendicularly to the axis 206 of the embodiment at the level L. As a skilled person now readily appreciates, in operation of the embodiment, the front surface of the so-formed composite (as discussed - containing two constituent parts) tip 140 is brought in contact with the surface of the cornea.

FIG. 3 complements the description of the embodiment 100 of FIG. 1A and provides additional structural details that may help the reader understand the nature of the front surface 304 of the embodiment 100 As discussed in US 10,463,251 for example, a relevant portion of the tip of the embodiment 100 is designed to be brought against the cornea of an eye and is shown in a partial cross-sectional view. A cornea-contacting surface 304 includes a central concave surface portion 304A, which is optionally adapted to and is congruent with the curvature of the cornea and which in a specific embodiment may include a surface that is rotationally symmetric about an axis 306. The example of FIG. 3 shows only such specific rotationally symmetric case. In operation, the central concave surface portion 304A is brought in contact with the surface of the cornea. In at least some specific implementations, at a periphery of the corneal contact surface 304 the central concave surface portion 304A passes over into a peripheral surface portion 304B that - when present - has a curvature of an opposite sign (as compared to that of the central surface portion 304A). The peripheral surface portion 304B defines a looped (and in the specific case - annular) projection, along the axis 306 and onto a plane transverse to the axis 306. The central concave surface portion 304A and the peripheral annular portion 304B (when present) are tangentially, seamlessly merging into each other along a closed curve defined in a plane that is tangential to the surface 304 and perpendicular to the axis 306.

Notably - and by analogy with the configuration of the outer, peripheral surface portion 304B of the front surface 304 of the embodiment 100 - the edge annularly-circumscribing the axis 206 surface of the rim 204 of the embodiment 138 can also be optionally made curved, with the curvature having a sign opposite to the sign of the curvature of the surface 202.

With the above-presented overview of typical configurations of the probes of interest, the skilled person will now appreciate the structures of embodiments of the covers that are intended to be juxtaposed with such probes.

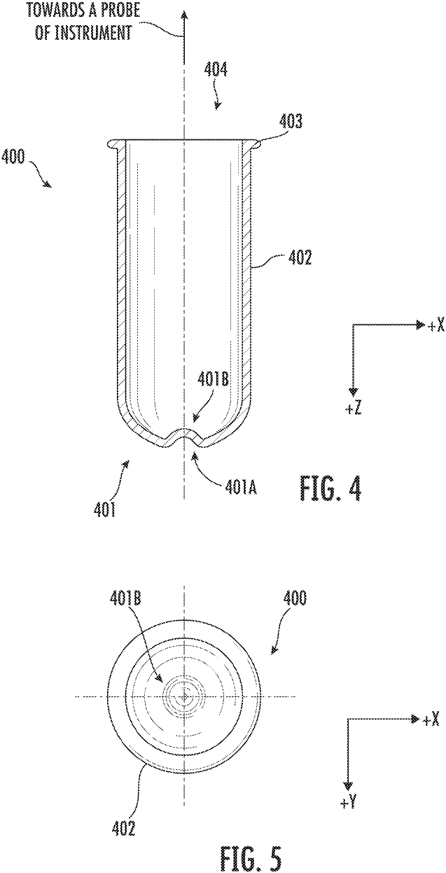

To this end, FIG. 4 is a cross-sectional view of an embodiment 400 of a prophylactic cover for a tip of a probe structured according to FIG. 1A and/or FIG. 1B and/or FIG. 1C. It is appreciated that embodiment 400 is a thin rubber (and, therefore, flexible) sheath which, when not folded in any way as shown in FIG. 4, has a generally tubular shape with one end closed and the other end open. (In that, the embodiment 400 in its unfolded form is substantially condom-like shaped, with the exception of the shape and structure of the very tip of the closed end, which is discussed below) The embodiment 400 has a tip area 401, at the closed end of the embodiment, characterized by a curved outer surface 401A and a curved inner surface 401B, and a wall 402 connecting the tip areas 401 with the open end 404 (the wall 402 forming the substantially cylindrical portion of the unfolded embodiment 400). The wall 402 may be complemented with a retention bead or ring bead 303 circumferentially formed at the wall 402 at the open end 404 of the cover 400. It is understood that, when the surface 401A is viewed in a -z direction, the surface 401A is perceived as a concave surface, and when the surface 301B is viewed in a +z direction (that is, internally to the cover, through the open end 404), the surface 301B is perceived as a concave surface. The curved shapes of the surfaces 401A and 401B in at least one specific case can be made substantially identical with one another such that the thickness of the cover remains substantially constant at every point at least within the bounds of the tip area 401. In a related implementation, at least the inner surface 401B may curved as described above while the outer surface 401A may be substantially flat.

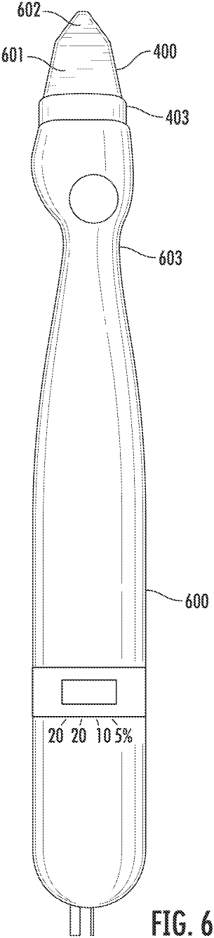

FIG. 5 schematically illustrates the cover 400 as viewed from the open end 404, in the +z direction, schematically showing the convex-like inner surface 401B of the sip area 401 and the optional circumferential retention bead or ring 403. It is understood that, in a given embodiment of the prophylactic cover, at least the inner convex surface 401B is preferably dimensioned to substantially conform to the shape of the front inwardly caved surface of the tip of the probe with which such cover is intended to be cooperated. (It is appreciated that when the target outer front surface of the tip of the probe is either substantially planar or even convex - as discussed, for example, in WO 2016/167827 - the inner surface shown as 401B in FIGS. 4 and 5 is preferably dimensioned to be substantially congruent with such target outer front surface of the tip, according to the idea of the present invention, to avoid the introduction of unnecessary and unpredictable errors during the corresponding measurement of a parameter of the eye with such a probe covered with the cover.)

FIG. 6 shows a representative contact tonometer 600 having a probe tip 601 that is configured to have a spatially curved eye-contacting surface (for example, configured according to the embodiments shown in FIGS. 1A, 1B or 1C) and having a measurement part (tip of the probe) 602 with an optionally disposable probe cover 400 shown positioned over the probe 601. The retention bead 303 may be appropriately dimensioned to hold the probe tip cover 300 onto the probe tip 501. In one example, a contact tonometer 600 is held by hand by medical personnel using the grip area 603 to perform the required test by contacting the contact front surface of the tip 602 of the probe 601 (which is covered by the tip cover 400) to the eye (cornea) of the patient. The disposable tip cover 400 protects the eye of the patient during the testing procedure.

It is appreciated that the scope of the invention also includes a judiciously-shaped cover for the conventionally-structured tip of a probe of a contact ophthalmological instrument -such as, for example, a tip the front cornea-contacting surface of which is substantially planar (the examples of such tip include the tip of the currently-employed in related art tonometer of a Tono-Pen® variety or the tip of the conventional ultrasound pachymeter.) The corresponding embodiment of the invention is a disposable article of manufacture that is dimensioned to cover a tip of a probe of a contact ophthalmological instrument and that includes a generally condom-shaped thin film body having an axis. Such body has a wall portion defining an open end of the article, and a tip portion defining a closed end of the article. The tip portion includes a first central area that is concave as viewed along the axis towards the open end but the inner surface of the first central area in this case is substantially planar.

The term “hypoallergenic” means and is defined as a characteristics representing a decreased tendency to cause an allergic reaction. The term “latex” means an aqueous emulsion of finely divided rubber or plastic particles. The term “synthetic rubber” means a rubbery material which does not contain any natural latex rubber. All weights, amounts and ratios herein are by weight, unless otherwise specifically noted.

The hypoallergenic disposable probe tip cover structured in accord with the idea of the invention a judiciously shaped article which is placed over the curved-front-surface probe tip of a chosen contact ophthalmological instrument tonometer to shield patients and medical personnel from cross-contamination. The probe tip cover, or sheath, is formed from a hypoallergenic material, and may be disposable after a single use. The cover fits over the judiciously-curved probe tip such as to form contact between the curved portion of the tip of the cover and the curved front surface of the probe tip substantially without gaps. The curved portion of the cover tip covers at least the measurement (curved) area of the probe tip and is held onto the probe by frictional forces or a combination of frictional forces and retention means.

In some embodiments, but not necessarily in all embodiments, optical transparency of the cover may be another desirable characteristic and is, therefore, employed. An embodiment of a practically useful hypoallergenic tip cover of the invention preferably has a thickness of less than about 0.010 inch, preferably from about 0.0005 inch to about 0.005 inch. Available synthetic elastomeric materials from which an embodiment may be made may include but are not limited to nitrile, polyurethanes, polyethylene, polypropylene, polyisoprene, polychloroprene, silicone, styrene-butadiene block copolymers, block copolymers including blocks formed from isoprene, ethylene butylenes, and/or styrene blocks, acrylonitrile-butadiene, polybutadiene and the like. Useful thin films formed from such elastomers are those having an ultimate elongation of from about 500% to 1000%, a tensile strength of from about 1000 psi to about 5500 psi, and a modulus of elasticity at 100% strain of from about 50 psi to about 2000 psi. In one embodiment, the thin film has a modulus of elasticity is from about 50 psi to about 500 psi.

In a related implementation, the disposable tip cover may be formed from synthetic cis-1,4-polyisoprene. Polyisoprene lacks the proteins in natural rubber that can be the basis of allergic reaction at the cornea. If processed in such a fashion so as not to introduce any irritants such as sulfur or similar proteins to those contained in natural rubber, they are hypoallergenic and have the required properties such as elongation and tensile strength. Polyisoprene polymers and copolymers useful to form probe tip covers of the invention have an elongation of at least about 800%, a tensile strength of at least about 2500 psi, and a modulus of elasticity having a value of from about 250 psi to about 500 psi at 100% strain.

Embodiments of the probe tip cover of the invention may be then formed by conventional film-forming means including molding, extrusion, blowing and casting of films, and lamination.

After formation/manufacture of the probe tip cover, removable holding means may be attached to the cover. One holding means may be configured as a cylinder that is wrapped around the probe tip cover. Such a removable holding means is used by the installer to place the probe tip cover onto the probe tip of the contact tonometer without contacting the sterile cover. The cylinder is then peeled from the cover without contacting the cover itself to maintain its sterile nature. The cylinder may be formed of a fibrous material such as cardboard, which is easy to grasp, and may include perforations to reduce the manual effort required to remove the cylinder from the cover. Alternative removable holding means includes such structures as peel-off flaps, tabs or flanges. If such removable holding means are not desired, the disposable tip cover may be provided e.g., in a rolled form, where it will be rolled down onto the probe tip and handled only by the lower wall area, or retention bead.

For the purposes of this disclosure and the appended claims, the expression of the type “element A and/or element B” has the meaning that covers embodiments having element A alone, element B alone, or elements A and B taken together and, as such, is intended to be equivalent to “at least one of element A and element B”.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention. Within this specification, embodiments have been described in a way that enables a clear and concise specification to be written, but it is intended and will be appreciated that embodiments may be variously combined or separated without parting from the scope of the invention. In particular, it will be appreciated that all features described herein at applicable to all aspects of the invention.

When the present disclosure describes features of the invention with reference to corresponding drawings (in which like numbers represent the same or similar elements, wherever possible), the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, at least for purposes of simplifying the given drawing and discussion, and directing the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this particular detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

Moreover, if the schematic flow chart diagram is included, the depicted order and labeled steps of the logical flow are indicative of one embodiment of the presented method. Other steps and order of steps may be conceived that are equivalent in function, logic, or effect to one or more steps, or portions thereof, of the illustrated method. Without loss of generality, the order in which processing steps or particular methods occur may or may not strictly adhere to the order of the corresponding steps shown.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. The use of this term in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated may vary within a range defined by an experimental measurement error that is typical when using a measurement method accepted in the art for such purposes. As an example only, a reference to a vector or line or plane being substantially parallel to a reference line or plane is to be construed as such vector or line extending along a direction or axis that is the same as or very close to that of the reference line or plane (with angular deviations from the reference direction or axis that are considered to be practically typical in the art, for example between zero and fifteen degrees, more preferably between zero and ten degrees, even more preferably between zero and 5 degrees, and most preferably between zero and 2 degrees). For example, the terms "approximately" and about", when used in reference to a numerical value, represent a range of plus or minus 20% with respect to the specified value, more preferably plus or minus 10%, even more preferably plus or minus 5%, most preferably plus or minus 2%. The terms “flexible” and “substantially flexible”, when used interchangeably in reference to a given object, generally identifies the structural nature of such object that is capable of bending easily without breaking, that is elastically pliable.

The invention as recited in claims appended to this disclosure is intended to be assessed in light of the disclosure as a whole, including features disclosed in prior art to which reference is made.

Although specific embodiments have been illustrated and described herein for purposes of description of the preferred embodiment, it will be appreciated by those of ordinary skill in the art that a wide variety of alternate and/or equivalent implementations calculated to achieve the same purposes may be substituted for the specific embodiments shown and described without departing from the scope of the present invention. This application is intended to cover any adaptations or variations of the preferred embodiments discussed herein. Accordingly, the invention should not be viewed as being limited to the disclosed embodiment(s).

What is claimed is:

1. A disposable article of manufacture dimensioned to cover a tip of a probe of a contact ophthalmological instrument, the article comprising:
a flexible thin film tubular body having an open and a closed end, a tip portion defining the closed end of the body, and a wall portion connecting the closed end with the open end,
wherein the tip portion includes a first central area that has an inner surface and an outer surface, the outer surface being concave,
wherein said article is configured to create a barrier, when installed onto the probe tip, to transfer of microorganism between an eye of a patient and the tip of a probe of the contact instrument during a contact ophthalmological examination while not impeding a measurement of a target parameter of the eye through said cover.

2. A disposable article according to claim 1, wherein:
(2A) the inner surface is either convex or substantially planar as viewed internally to tubular body from the open end; or
(2B) the inner surface is convex;
and wherein the concave outer surface is dimensioned to substantially conform to a corneal surface of an eye.

3. A disposable article according to claim 1, wherein said open end is circumscribed with a retention ring portion of the article.

4. A disposable article according to claim 1, wherein one of the following conditions is satisfied:
(4A) when the inner surface of the first central area is convex,
said article is dimensioned for use with a contact tonometer employing a tonometer probe having a tip with an inwardly curved front surface,
the convex inner surface of the first central area is substantially congruent with the inwardly curved front surface,
the first central area is dimensioned to substantially cover the inwardly curved surface, and
the target parameter includes the intraocular pressure (IOP);
(4B) when the inner surface of the first central area is substantially planar,
said article is dimensioned for use with a contact tonometer employing a tonometer probe tip having a substantially planar front surface of the tonometer probe tip,
the first central area is dimensioned to substantially cover the front surface of the tonometer probe tip, and
the target parameter includes the IOP; and
(4C) said article is configured for use with a contact ultrasound pachymeter employing a pachymeter probe having a tip with an inwardly curved cornea-contacting surface,
wherein the inner surface of the first central area is dimensioned to cover and substantially conform to said cornea-contacting surface of the tip of the pachymeter probe.

5. A disposable article according to claim 1, wherein the wall portion is dimensioned to secure said cover to said contact ophthalmological instrument.

6. A disposable article according to claim 3, wherein said retention ring is configured to compressingly fit into a corresponding groove of said contact instrument.

7. A disposable article according to claim 1, wherein, when the outer surface of the first central area the tip portion is necessarily concave, the tip portion further includes a second peripheral area having an outer surface that is convex, the second peripheral area circumscribing the first central area.

8. A disposable article according to claim 1, configured to have:
an ultimate elongation from about 500% to about 1000%, and/or
a tensile strength from about 1000 to about 5500 psi, and/or
a modulus of elasticity, at 100% strain, from about 50 to about 2000 psi.

9. A disposable article according to claim 1, wherein the thin film is formed from a material selected from the group consisting of polyurethane, polyethylene, polypropylene, polyisoprene, polychloroprene, nitrile, and silicone.

10. A method comprising:
removably covering a tip of a probe of a contact ophthalmological instrument with the disposable article of manufacture according to claim 1 to spatially-coordinate the first central area with a front surface of the tip of a probe; and
bringing the tip of the probe in indirect contact with the cornea of an eye through a layer of the thin film body of the article at the first central area such that an outer surface of the first central area is in physical contact with the cornea while the tip of the probe is in physical contact with an inner surface of the first central area.

11. A method according to claim 10, wherein said bringing includes bringing substantially congruent with one another inner surface of the first central area and the front surface of the tip of the probe in contact with one another.

12. A method according to claim 10, wherein said covering includes creating a barrier against microorganisms from the eye to said tip of a probe during said indirect contact while allowing a measurement of a target parameter of the eye through said cover substantially without impeding said measurement.

13. A method according to claim 10, satisfying one of the following conditions:
(13A) wherein the inner surface of the first central area is convex as viewed from an open end of the disposable article;
wherein the tip of the probe has an inwardly curved front surface, and
wherein said covering includes substantially conforming said inner surface of the tip portion of a thin film body of the disposable article to the inwardly curved front surface of the tip of the probe, and
(13B) wherein the tip of the probe has a substantially planar front surface, and
wherein said covering includes substantially conforming said inner surface of the tip portion of the thin film body to the inwardly curved front surface of the tip of the probe.

14. A method according to claim 10, wherein said removably covering includes securing the disposable article on the ophthalmological instrument by placing a retention ring portion of the article into a groove on said instrument.

15. A method according to claim 10, further comprising:
(15A) performing a measurement of an intraocular pressure of an eye while pressing the tip of the probe and the covering into the cornea; or
(15B) performing a measurement of a thickness of the cornea by transmitting ultrasound waves through said covering while the covering remains in contact with the cornea.
